(19) United States
(12) Patent Application Publication
(10) Pub. No.: US 2021/0108172 A1
(43) Pub. Date: Apr. 15, 2021

(54) THREE DIMENSIONAL HYDROGEL FOR CULTURING OF CELLS

(71) Applicant: Regents of the University of Minnesota, Minneapolis, MN (US)

(72) Inventors: Timothy D. O'Brien, Minneapolis, MN (US); John H. Brekke, Minneapolis, MN (US); Aubie Shaw, Minneapolis, MN (US)

(21) Appl. No.: 17/070,368

(22) Filed: Oct. 14, 2020

Related U.S. Application Data
(60) Provisional application No. 62/914,846, filed on Oct. 14, 2019.

(51) Int. Cl. C12N 5/00; C12N 5/077; C12N 5/074; A61K 35/32

(52) U.S. Cl. CPC C12N 5/0062; C12N 5/0655; C12N 2513/00; A61K 35/32; C12N 2506/1353; C12N 5/0696

(57) ABSTRACT

Fully dissolved, aqueous solutions of polycationic macromolecules and polyanionic macromolecules with a cell suspension are mixed to form a three dimensional hydrogel, useful for clinical, diagnostic, and therapeutic applications.

### CROSS REFERENCE TO RELATED APPLICATIONS

This non-provisional application claims the benefit of priority to U.S. Provisional Application No. 62/914,846, filed 14 Oct. 2019, which is incorporated by reference in its entirety.

### FIELD OF THE INVENTION

The present disclosure relates to methods of preparing a three dimensional hydrogel embedded with cells and/or a therapeutic/biologically-active agent from separate solutions of polycations and polyanions.

### BACKGROUND

Traditional in vitro cell culture methods employ two dimensional, polystyrene cell culture plates. While much valuable information has been discovered in such systems, two significant issues limit their application to tissue engineering problems. First, in vitro culture plates are generally rigid structures made, typically, of polystyrene. Secondly, in vitro culture plates provide the cells with only a two dimensional substratum. This two dimensional substratum is restrictive, since cells of tissues and organs respond to signals initiated within a three dimensional (3-D) microenvironment. Cells comprising all tissues have some capacity to alter their three dimensional morphologies in response to changes in mechanical forces present within their environments. 3-D hydrogel constructs have been formed by blending small dry particles of polycationic and polyanionic macromolecules in specific mass ratios and subsequently exposing these dry particles to a hydrating fluid that also functions as a cell suspension solution (Lindborg, B A et al, (2015) Tissue Engineering: Part A, 21 (11 and 12):1952-1962). When dissolved in the hydrating fluid, these macromolecules are electrostatically joined, forming insoluble, polyelectrolytic complex fibers in addition to networks of intermolecular and intramolecular hydrogen bonds (U.S. Pat. Nos. 7,524,514; 8,137,696). The polyanion macromolecule (hyaluronan) was used in a fully dissolved condition in the hydration/cell suspension fluid while maintaining the polycation macromolecule (chitosan) in the condition of a dry, 3-D architecture, possessed of intercommunicating void spaces. (U.S. Pat. No. 9,981,067).

### SUMMARY

The invention provides hydrogel compositions useful for cell culture methods and/or delivery of therapeutic/biologically-active agents, as well as processes for their preparation where both polycationic and polyanionic macromolecules are fully dissolved in separate volumes of aqueous media, such as sterile water, as a first step and are then contacted while fully dissolved in their respective solutions. The hydrogel construct is generated by spontaneous electrostatic association of the cationic and anionic macromolecules, as well as by spontaneous formation of intra-molecular and intermolecular hydrogen bonds. No crosslinking agents are necessary. The entire three dimensional (3-D) construct may be formed in the presence of a suspension of cells in a chamber, thus customizing the hydrogel to individual cells (or clusters of cells) as it is being generated by hydrogen bonding and polyelectrolytic union forces of the macromolecules.

An aspect of the invention is a process for preparing a hydrogel comprising contacting a fully dissolved, aqueous solution of at least one polycationic macromolecule with a fully dissolved, aqueous solution of at least one polyanionic macromolecule in a chamber whereby the hydrogel is formed.

An aspect of the invention are methods of preparing a three dimensional hydrogel embedded with cells and/or therapeutic or biologically-active agent(s) from separate solutions of polycations and polyanions. In certain embodiments, the polycationic and polyanionic solutions may be fabricated together with a porous fabric, such as 3-dimensional architectures of D,D-L,L polylactic acid, employed as depots for other therapeutic/biologically-active agents not readily dissolved in water-based solutions, such as chemotherapeutic compounds useful for high concentration, regional therapy of malignant tumors.

Thus, in certain embodiments, the fully dissolved, aqueous solution of at least one polyanionic macromolecule further comprises a suspension of cells. In certain embodiments, the fully dissolved, aqueous solution of at least one polycationic macromolecule further comprises a suspension of cells. In certain embodiments, the process further comprises contacting the prepared hydrogel with a plurality of cells.

In certain embodiments, the fully dissolved, aqueous solution of at least one polyanionic macromolecule further comprises at least one therapeutic/biologically-active agent co-solubilized with the polyanion. In certain embodiments, the fully dissolved, aqueous solution of at least one polycationic macromolecule further comprises at least one therapeutic/biologically-active agent solubilized with the polycation. In certain embodiments, the coacervate generated by blending the polyanionic and polycationic solutions together may be conjoined with a porous fabric, such as 3-dimensional architectures of D,D L,L polylactic acid, which may be employed as depots for at least one therapeutic/biologically-active agent (e.g., an agent that is not readily dissolved in water-based solutions, such as chemotherapeutic compounds useful for high concentration, regional therapy of malignant tumors). In certain embodiments, the fully dissolved, aqueous solution of at least one polyanionic macromolecule further comprises at least one therapeutic/biologically-active agent co-solubilized with the polyanion, the fully dissolved, aqueous solution of at least one polycationic macromolecule further comprises at least one therapeutic/biologically-active agent solubilized with the polycation, and/or the porous fabric comprises at least one therapeutic/biologically-active agent.

Another aspect of the invention is a process for preparing a three dimensional hydrogel embedded with cells comprising contacting a fully dissolved, aqueous solution of at least one polycationic macromolecule with a fully dissolved, aqueous solution of at least one polyanionic macromolecule comprising a suspension of cells in a chamber whereby the three dimensional hydrogel is formed.

Another aspect of the invention is a process for preparing a three dimensional hydrogel embedded with cells comprising contacting a fully dissolved, aqueous solution of at least one polycationic macromolecule comprising a suspension of cells (e.g., bacterial cells) with a fully dissolved, aqueous solution of at least one polyanionic macromolecule in a chamber whereby the three dimensional hydrogel is formed.

Another aspect of the invention is a process for preparing a three dimensional hydrogel comprising at least one therapeutic/biologically-active agent, the process comprising contacting a fully dissolved, aqueous solution of at least one polycationic macromolecule with a fully dissolved, aqueous solution of at least one polyanionic macromolecule comprising at least one therapeutic/biologically-active agent in a chamber whereby the three dimensional hydrogel is formed.

Another aspect of the invention is a process for preparing a three dimensional hydrogel comprising at least one therapeutic/biologically-active agent, the process comprising contacting a fully dissolved, aqueous solution of at least one polycationic macromolecule comprising at least one therapeutic/biologically-active agent with a fully dissolved, aqueous solution of at least one polyanionic macromolecule in a chamber whereby the three dimensional hydrogel is formed.

Another aspect of the invention is a process for preparing a three dimensional hydrogel comprising at least one therapeutic/biologically-active agent, the process comprising contacting a fully dissolved, aqueous solution of at least one polycationic macromolecule; a fully dissolved, aqueous solution of at least one polyanionic macromolecule; and a porous fabric comprising at least one therapeutic/biologically-active agent, in a chamber, whereby the three dimensional hydrogel is formed.

Another aspect of the invention is a method for monitoring cell behavior comprising observing by microscopy (e.g., light microscopy) a suspension of cells in a three dimensional hydrogel prepared by a process comprising contacting (e.g., mixing) a fully dissolved, aqueous solution of a polycationic macromolecule with a fully dissolved, aqueous solution of a polyanionic macromolecule comprising the suspension of cells in a chamber whereby the three dimensional hydrogel is formed.

Another aspect of the invention is a method for monitoring cell behavior comprising observing by microscopy (e.g., light microscopy) a suspension of cells in a three dimensional hydrogel prepared by a process comprising contacting (e.g., mixing) a fully dissolved, aqueous solution of a polycationic macromolecule comprising a suspension of cells with a fully dissolved, aqueous solution of a polyanionic macromolecule in a chamber whereby the three dimensional hydrogel is formed.

Another aspect of the invention is a hydrogel composition prepared by contacting a fully dissolved, aqueous solution of a polycationic macromolecule with a fully dissolved, aqueous solution of a polyanionic macromolecule in a chamber whereby the hydrogel is formed. In certain embodiments, the chamber contains a porous fabric. In certain embodiments, the at least one polycationic macromolecule fills interstices of the porous fabric.

In certain embodiments, the hydrogel composition further comprises a plurality of cells (e.g., embedded in the hydrogel or otherwise in contact with the hydrogel). Thus, in certain embodiments, the fully dissolved, aqueous solution of at least one polyanionic macromolecule further comprises a suspension of cells. In certain embodiments, the fully dissolved, aqueous solution of at least one polycationic macromolecule further comprises a suspension of cells.

In certain embodiments, the hydrogel composition further comprises at least one therapeutic/biologically-active agent (e.g., embedded in the hydrogel or otherwise in contact with the hydrogel). Thus, in certain embodiments, the fully dissolved, aqueous solution of at least one polyanionic macromolecule further comprises at least one therapeutic/biologically-active agent. In certain embodiments, the fully dissolved, aqueous solution of at least one polycationic macromolecule further comprises at least one therapeutic/biologically-active agent. In certain embodiments, the porous fabric comprises at least one therapeutic/biologically-active agent.

Another aspect of the invention is a three dimensional hydrogel composition prepared by contacting a fully dissolved, aqueous solution of a polycationic macromolecule with a fully dissolved, aqueous solution of a polyanionic macromolecule comprising a suspension of cells in a chamber whereby the three dimensional hydrogel is formed.

Another aspect of the invention is a three dimensional hydrogel composition prepared by contacting a fully dissolved, aqueous solution of a polycationic macromolecule comprising a suspension of cells with a fully dissolved, aqueous solution of a polyanionic macromolecule in a chamber whereby the three dimensional hydrogel is formed.

Another aspect of the invention is a three dimensional hydrogel composition prepared by contacting a fully dissolved, aqueous solution of a polycationic macromolecule with a fully dissolved, aqueous solution of a polyanionic macromolecule comprising at least one therapeutic/biologically-active agent in a chamber whereby the three dimensional hydrogel is formed.

Another aspect of the invention is a three dimensional hydrogel composition prepared by contacting a fully dissolved, aqueous solution of a polycationic macromolecule comprising at least one therapeutic/biologically-active agent with a fully dissolved, aqueous solution of a polyanionic macromolecule in a chamber whereby the three dimensional hydrogel is formed.

Another aspect of the invention is a three dimensional hydrogel composition prepared by contacting a fully dissolved, aqueous solution of a polycationic macromolecule; a fully dissolved, aqueous solution of a polyanionic macromolecule; and a porous fabric comprising at least one therapeutic/biologically-active agent, in a chamber whereby the three dimensional hydrogel is formed.

Another aspect of the invention is a method of three dimensional cell culture comprising growing cells in a three dimensional hydrogel composition prepared by a process described herein.

Another aspect of the invention provides a three dimensional hydrogel composition described herein for use in medical therapy.

Another aspect of the invention is a method of treating a disease, disorder or condition in a mammal in need thereof, comprising administering to the mammal a three dimensional hydrogel composition described herein.

Another aspect of the invention is a three dimensional hydrogel composition described herein for use in the treatment of a disease, disorder or condition in a mammal in need thereof.

Another aspect of the invention is the use of a three dimensional hydrogel composition described herein to prepare a medicament for the treatment of a disease, disorder or condition in a mammal in need thereof.

In certain embodiments, the disease, disorder or condition is selected from the group consisting of degenerative disc disease (DDD), traumatic articular cartilage injuries, osteoarthritis, periodontal defects, bone defects, and diabetes mellitus.

In certain embodiments, the disease, disorder or condition is cancer.

Another aspect of the invention is a method of delivering a therapeutic/biologically-active agent to a mammal in need thereof, comprising administering to the mammal a three dimensional hydrogel composition described herein, which comprises a therapeutic/biologically-active agent.

### BRIEF DESCRIPTION OF THE DRAWINGS

FIG. 1 shows a scanning electron microscope (SEM) image of a cross-section of Epi-Guide® (Curasan Inc.) fabric showing graded pore volumes. The largest and least dense organization shown at top surface; smallest and most dense organization to bottom surface demonstrating a cell barrier function.

FIG. 2 shows a scanning electron microscope (SEM) image of Epi-Guide® (Curasan Inc.) fabric from top surface with large pore configuration.

FIG. 3 shows a scanning electron microscope (SEM) image of Epi-Guide® (Curasan Inc.) fabric from bottom surface with small pore configuration.

FIG. 4 shows a 10× microscope image of a hydrogel construct mounted vertically on a cover slip.

FIG. 5A shows a microscope image of BX40-200x Epi-Fluorescence at 610 nm. Chitosan treated with Q-dots fluorescing at 610 nm. DMEM (Dulbecco's Modified Eagle's Medium)-Hyaluronan does not fluoresce but is back-lit by fluorescence from adjacent Q-dot treated.

FIG. 5B shows a microscope image of BX40-200x Epi-Fluorescence at 610 nm and the same image as shown in FIG. 5A, but annotated here to identify elements which may be HA-CT-PEC (hyaluronan-chitosan-polyelectrolyte complex) filaments originating at interface of chitosan solution within Epi-Guide® (Curasan Inc.) interstices and DMEM/hyaluronic acid/cell suspension solution, laminated on surface of Epi-Guide®/chitosan complex.

FIG. 6 shows a microscope image of BX40-100x Epi-Fluorescence at 610 nm. The surface of DMEM/hyaluronic acid/cell suspension hydrogel component shows suspected projection of HA-CT-PEC filaments (polyelectrolyte complex) at DMEM surface.

FIG. 7 shows a microscopy image of iPSC (induced pluripotent stem cells) cultured in a DMEM/hyaluronic acid/cell suspension hydrogel and H&E stained (hematoxylin and eosin) less than 60 minutes out of fresh media according to Example 6.

FIG. 8 shows a microscopy image of iPSC cultured in a DMEM/hyaluronic acid/cell suspension hydrogel and H&E stained (hematoxylin and eosin) greater than 60 minutes out of fresh media according to Example 6.

FIG. 9 shows a 10× microscopy image of iPSC cultured according to Example 5, lifted from Well #1A and deposited intact on a cover slip after 36 hours reaction.

FIG. 10 shows a 20× microscopy image of iPSC cultured according to Example 5, of a sheet raised from Well #1A, saturated with media after 36 hours reaction.

### DEFINITIONS

The term “stiffness” refers to a composition's ability to resist deformation in response to an applied force. As used herein, the term “stiffness” describes a composition's elastic modulus and is defined by the ratio of stress (force applied per unit area along an axis) over strain (degree of deformation over initial length along that axis). Stiffness also means tensegrity, tensional integrity, or floating compression.

The term “polyelectrolyte: refers to a polymer whose repeating units bear an electrolyte group. Polycations are positively charged and polyanions are negatively charged, both are polyelectrolytes.

The term “hydrogel” refers to a hydrophilic composition of a network of polymers. Hydrogels also possess a degree of flexibility very similar to natural tissue, due to their significant water content. Hydrogel includes a hydrocolloid, including a three dimensional hybrid hydrocolloid-hydrogel composition.

The term “hydrocolloid” refers to a composition of high molecular weight polymers containing charged or polar groups rendering them hydrophilic. A colloid is a substance microscopically dispersed evenly throughout another substance. A hydrocollloidal system consists of two separate phases: a dispersed phase (or internal phase) and a water based continuous phase (or dispersion) medium.

The term “coacervate” refers to an aqueous phase rich in macromolecules such as synthetic polymers, proteins or nucleic acids. A coacervate forms through liquid-liquid phase separation (LLPS) leading to a dense phase in thermodynamic equilibrium with a dilute phase.

The term “irreversible hydrocolloid” refers to a hydrocolloid the physical state of which is changed by an irreversible chemical reaction when water is added to a powder and an insoluble substance is formed.

The term “therapeutic agent” refers to a molecule or chemical compound formulated as a treatment for a specific disease or biologic malady.

The term “biologically-active agent” refers to a molecule or chemical compound, including but not limited to a protein, formulated to interact with naturally occurring, biologic processes to enhance or redirect their natural actions.

### Polyelectrolytes of the Invention

Chitosan (CAS Reg. No. 9012-76-4) is a linear polysaccharide composed of randomly distributed β-linked D-glucosamine and N-acetyl-D-glucosamine. It is made by treating the chitin shells of shrimp and other crustaceans with an alkaline substance, like sodium hydroxide. Chitin is the major structural constituent of the exoskeleton of crustaceans and insects and is a component of the cell wall of fungi. Chitosan (poly-beta₁₋₄-glucosamine) is the highly deacetylated form of chitin and is classified as an amino polysaccharide. Chitosan (CT) is protonated by exposure to either mineral or organic acid and is thus rendered soluble. Degree of CT protonation is related to solubility. A minimum of about 45% protonation of the available amine groups renders CT soluble. Preferably, chitosan is protonated from about 45% to about 100%. When exposed to pH levels below about 5.0, the amine groups (—NH₂) of chitosan molecules become protonated to ammonium, thus rendering the molecules soluble in water and providing them with a strong positive charge (cation) that attracts negatively charged molecules (anions). Protonation of between about 45% and about 100% of available amine groups may be controlled to modify degree of interaction with the anion. At certain degrees of protonation, chitosan may be thought of as an amphoteric composition, since it may both accept and donate protons, although chitosan is traditionally thought of as a cation in aqueous solution.

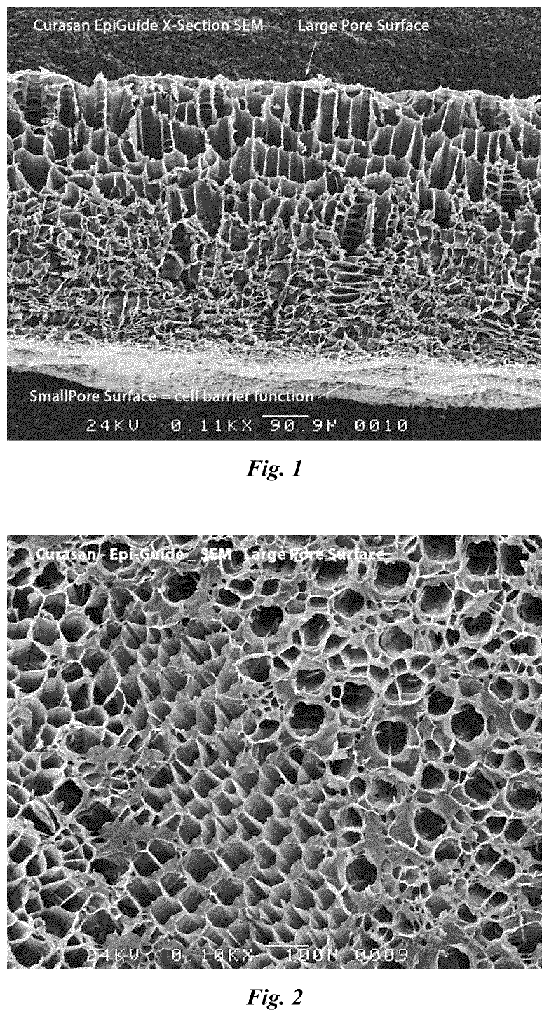

Protonation may occur by the exposure of chitosan to an acid to form a solution, preferably the substantially stoichiometric addition of an acid. The acid may be any inorganic or organic acid, preferably formic acid or glacial acetic acid. After the chitosan is protonated by exposure to an acid, the protonated CT solution may be lyophilized to a stiff porous chitosan fabric. The architecture of this CT fabric may be that of randomly sized, randomly shaped, intercommunicating interstices. CT fabric dimensions and physical properties may be controlled by the concentration of protonated CT in solution. The lyophilized CT fabric may be reduced to individual leaflets or platelets of dry protonated chitosan. These small pieces of CT fabric may have a thickness of about 1-10 micrometers and irregular planar shapes and dimensions. FIG. 1 represents a dry chitosan leaflet prepared according to one method of the present invention.

Chitosan may be obtained with a wide range of molecular weights from about 600,000 to 900,000 Da (Primex EHF, Iceland). However, it is recognized that greater or lesser Mw examples of CT may be employed to accommodate specific biologic requirements of cells to be cultured within its hydrogel or meet specific mechanical demands required of the final device. Further, chitosan exhibits interesting biological properties that may be used clinically. It is hemostatic and cicatrizing and may be used as a cell culture support. Its antimicrobial capacity acts by stimulation of the immune system and, in particular, it induces the activation of macrophages.

In some embodiments, the polycationic macromolecule comprises chitosan. Chitosan offers several advantageous biologic properties in support of cell implantation. Chitosan has inherent antimicrobial properties to prevent growth of gram-negative and gram positive bacteria, as well as fungi. Chitosan has varying effects on the innate immune system based on its degree of deacetylation. At deacetylation levels below 90%, chitosan may activate the innate immune system through ficolins which activate the lectin pathway of the complement system. When deacetylation levels are greater than 90%, circulating ficolins do not recognize chitosan and the complement system and immune systems are not activated. Protonated amine groups of chitosan chelate cationic Zn(II) moieties of matrix metalloproteases (MMPs) thus inhibiting MMP destructive activities (as, for example, in osteoarthritis). Previous hybrid gel compositions have chitosan at 85-87.5% degrees of deacetylation.

Deacetylation of chitosan may also result in an increase of primary amines, thus changing the pKa of its protonated amine groups, and altering the degree of ionization of protonated amine groups as a function of environmental pH. Chitosan has a pKa of about 6.5. Variation of chitosan's pKa may facilitate the formation process of the composition when fully hydrated and act as a buffering system to maintain environmental pH at acceptable physiologic levels. In some embodiments, the pKa may be decreased due to increased deacetylation of chitosan. In some embodiments, chitosan may be deacetylated to at least 60%, at least 70%, at least 80%, at least 90%, at least 100%, or a range between any of these values. In certain embodiments, chitosan may be deacetylated to at least 90%. In other embodiments, chitosan may be deacetylated to 100%. In such embodiments, chitosan may be protonated to the degree of about 45% to about 100% of available amine groups, about 50% to about 90% of available amine groups, about 60% to about 80% of available amine groups, and any percentage in between any of these values (including endpoints).

Hyaluronic acid (CAS Reg. No. 9004-61-9), also known as “hyaluronan,” is a linear polyanionic polysaccharide, and is a member of the family known as glycosaminoglycans. It is present in most vertebrate connective tissues at relatively high concentrations (up to 10 mg/ml). Hyaluronic acid (HY) may be obtained with an original weight average molecular weight of about 1.8×10⁶ Da. Upon size reduction to small particles, the Mw may be reduced to 1.0×10⁶ Da. As with its chitosan partner in this construct, the molecular weight of HY may be altered to accommodate specific biologic demands of cells to be cultured. The basic structural unit of HY is a disaccharide consisting of D-glucuronic acid (GlcA) in beta 1-3 linkage to N-acetyl-D-glucosamine (GlcNAc). The disaccharides may be linked together in a beta 1-4 linkage. In its highly hydrated form, hyaluronic acid shows unique properties of viscoelasticity and plasticity.

In cartilage, hyaluronic acid plays a central role in the assembly and maintenance of the macromolecular components constituting the chondrocytes' extracellular matrix (ECM). It binds with high specificity and affinity to aggregate and link protein. A single hyaluronic acid chain may form a central “filament” that binds a large number of aggregant molecules, forming a supermolecular complex that immobilizes water and leads to a highly hydrated gel-like structure. In addition, hyaluronic acid binds with high affinity to the CD44 receptor which is expressed on many cell types including, for instance, chondrocytes. Hyaluronic acid is naturally present in the vitreous humor of the eye and in the synovial fluid of joint cavities. It is used in surgical procedures involving the anterior chamber of the eye, such as corneal transplants and the removal and replacement of a cataractous lens. It is also used in the therapy of arthritis where injection of hyaluronic acid into the joint space may restore the rheological properties of the synovial fluid.

In its naturally occurring form hyaluronic acid is a salt, such as, for example, a sodium salt. The naturally occurring form may be subject to an ion exchange process to convert hyaluronic acid to an acid form. Although the sodium salt form is preferred, both forms of hyaluronic acid may be used. Changing the naturally occurring form to an acid form may change the traditionally anionic hyaluronic acid to a more amphoteric substance, being able to both accept and donate protons in aqueous solution.

Additional components may be added to the solvating fluid to promote cell viability and growth or direct pluripotent cell differentiation toward a particular phenotype. These additional components may include, for example, autologous and non-autologous serum and serum components, such as fetal bovine serum, albumin, growth factors, morphogens, hormones, cytokines, vitamins, and amino acids, et alia; as well as tissue specific growth factors; morphogens; dextran sulfate, such as, high molecular weight dextran sulfate and low molecular weight dextran sulfate; glycerol phosphate; normal saline; peptides that specifically bind an .alpha.5.beta.1 integrin, and products of the regenerating (Reg) gene super-family such as the islet neogenesis-associated growth protein (INGAP; reg). Furthermore, in some embodiments, the solvating fluid may have autologous interstitial fluid. In some embodiments, the solvating fluid may contain extracellular matrix glycoproteins and proteoglycans.

In some embodiments, a composition may comprise a plurality of polycationic macromolecules and a plurality of polyanionic macromolecules. Examples of polyanionic macromolecules include, but are not limited to, dextran sulfate and glycosaminoglycans, such as dermatan sulfate, keratan sulfate, heparan sulfate, and hyaluronan, or a combination thereof. For example, the plurality of polyanionic macromolecules may include dextran sulfate and an additional glycosaminoglycan, such as hyaluronan. In these embodiments, the dextran sulfate may have a low molecular weight of about 5 kilodaltons. The plurality of polycationic macromolecules may include, but are not limited to, cellulose, chitosan, any other linear polysaccharide capable of being protonated, or a combination thereof. For example, the plurality of polycationic macromolecules may include chitosan and cellulose.

In some embodiments, dextran sulfate may be used as the polyanionic macromolecule to mix with a polycationic macromolecule to form a hydrogel. In some embodiments, chitosan may be used as the polycationic macromolecule. In such embodiments, dextran sulfate electrostatically interacts with chitosan. Where a second polyanionic macromolecule may be used with a primary polyanionic macromolecule, the second polyanionic macromolecule may be a glycosaminoglycan. In some embodiments, such glycosaminoglycans may include hyaluronan, operating independently as a polyanionic macromolecule or as a companion (polyanionic macromolecule) to dextran sulfate.

Dextran sulfate, a polyanionic macromolecule, provides unique physical and biologic properties that contribute valuable structural and mechanical properties for the composition. Dextran sulfate has a specific molecular morphology providing the molecule with a high level of physical flexibility. Dextran sulfate comprises glucose molecules having three axes of rotation about .alpha.-1/6 glycosidic linkages uniformly joining the glucose molecules. Dextran sulfate also has a low persistence length (Lₚ) value of 1.6 nm, indicating low stiffness as well as high flexibility. As a result, dextran sulfate may efficiently associate with polycationic macromolecules as well as with cell surface receptors. For example, when dextran sulfate is reacted with chitosan, polyelectrolytic complexes (PEC) of the two macromolecules form by an electrostatic union of dextran sulfate's ROSO₃⁻ groups with NH₃+(ammonium) groups of chitosan molecules. These insoluble PEC fibers function as the composition's dispersed phase while providing structural and mechanical competency for its three-dimensional architecture.

### Certain Hydrogels of the Invention

A three dimensional hydrogel composition of the invention is prepared by a process of contacting a fully dissolved, aqueous solution of at least one polycationic macromolecule with a fully dissolved, aqueous solution of at least one polyanionic macromolecule in a chamber whereby the three dimensional hydrogel is formed.

In certain embodiments, the fully dissolved, aqueous solution of at least one polyanionic macromolecule further comprises a suspension of cells. In certain embodiments, the fully dissolved, aqueous solution of at least one polycationic macromolecule further comprises a suspension of cells. In an exemplary embodiment, the process includes wherein the suspension of cells is a cell culture comprising one or more extracellular matrix compounds selected from fibronectin, vitronectin, laminin, collagen type I, chondoitin sulfate, collagen type II, hyaluronan, dextran sulfate and a soluble signaling factor selected from growth factors, morphogens and cytokines.

In certain embodiments, the fully dissolved, aqueous solution of at least one polyanionic macromolecule further comprises at least one therapeutic/biologically-active agent. In certain embodiments, the fully dissolved, aqueous solution of at least one polycationic macromolecule further comprises at least one therapeutic/biologically-active agent.

In an exemplary embodiment, the process includes wherein the chamber contains a porous fabric, such as Epi-Guide® (Curasan Inc.), or other porous scaffolds composed of D,D, L,L polylactic acid, gelatin, methacrylate polymer, type I or type II collagen, hyaluronan, polyethylene glycol, polyester, polycarbonate, poly(α-hydroxy) acid, or other suitable materials.

In certain embodiments, the porous fabric comprises at least one therapeutic/biologically-active agent.

In an exemplary embodiment, at least one polycationic macromolecule fills interstices of the porous fabric.

In an exemplary embodiment, the process includes wherein the polycationic macromolecule is chitosan.

In an exemplary embodiment, the process includes wherein the polyanionic macromolecule is selected from the group consisting of hyaluronic acid, type I collagen, type II collagen, and dextran sulfate.

In an exemplary embodiment, the process includes wherein the fully dissolved, aqueous solution of at least one polyanionic macromolecule comprising a suspension of cells further comprises a cell culture media selected from DMEM or other suitable media.

In an exemplary embodiment, the process includes wherein the fully dissolved, aqueous solution of at least one polycationic macromolecule is deposited on the bottom surface of the chamber followed by adding the fully dissolved, aqueous solution of the at least one polyanionic macromolecule. In certain embodiments, the fully dissolved, aqueous solution of at least one polyanionic macromolecule comprises a suspension of cells. In certain embodiments, the fully dissolved, aqueous solution of at least one polyanionic macromolecule comprises at least one therapeutic/biologically-active agent.

In an exemplary embodiment, the process further comprises depositing a polycationic macromolecule to form a thin, dry film on the bottom surface of the chamber before contacting the fully dissolved, aqueous solution of at least one polycationic macromolecule with the fully dissolved, aqueous solution of at least one polyanionic macromolecule comprising a suspension of cells and/or at least one therapeutic/biologically-active agent, in the chamber. In certain embodiments, the thin, dry film may be treated with fluorescent Q-dots.

In an exemplary embodiment, the process includes wherein the fully dissolved, aqueous solution of the at least one polyanionic macromolecule comprising a suspension of cells and/or at least one therapeutic/biologically-active agent is added by injection into the at least one fully dissolved, aqueous solution of the polycationic macromolecule.

In an exemplary embodiment, the process includes wherein at least one polyanionic macromolecule comprising a suspension of cells and/or at least one therapeutic/biologically-active agent is added by injection with a needle.

In an exemplary embodiment, the process includes wherein the aqueous solution of the at least one polycationic macromolecule is treated with a thin, reinforcing matrix selected from D,D-L,L polylactic acid, collagen, and hyaluronic acid before adding the fully dissolved, aqueous solution of the at least one polyanionic macromolecule comprising a suspension of cells. In an exemplary embodiment, the process includes wherein the aqueous solution of the at least one polycationic macromolecule is treated with a thin, reinforcing matrix selected from D,D-L,L polylactic acid, collagen, and hyaluronic acid before adding the fully dissolved, aqueous solution of the at least one polyanionic macromolecule comprising at least one therapeutic/biologically-active agent.

In an exemplary embodiment, the process includes wherein the fully dissolved, aqueous solution of the at least one polyanionic macromolecule is deposited on the bottom surface of a chamber followed by adding the fully dissolved, aqueous solution of the at least one polycationic macromolecule. In certain embodiments, the fully dissolved, aqueous solution of at least one polyanionic macromolecule comprises a suspension of cells. In certain embodiments, the fully dissolved, aqueous solution of at least one polyanionic macromolecule comprises at least one therapeutic/biologically-active agent.

In an exemplary embodiment, the process further comprises depositing a polyanionic macromolecule to form a thin, dry film on the bottom surface of the chamber before contacting the fully dissolved, aqueous solution of at least one polycationic macromolecule with the fully dissolved, aqueous solution of at least one polyanionic macromolecule comprising a suspension of cells and/or at least one therapeutic/biologically-active agent, in the chamber. In certain embodiments, the thin, dry film may be treated with fluorescent Q-dots.

In an exemplary embodiment, the process includes wherein the fully dissolved, aqueous solution of the at least one polycationic macromolecule is added by injection into the at least one fully dissolved, aqueous solution of the polyanionic macromolecule comprising a suspension of cells and/or at least one therapeutic/biologically-active agent.

In an exemplary embodiment, the process includes wherein the fully dissolved, aqueous solution of at least one polycationic macromolecule is added by injection with a needle.

In an exemplary embodiment, the cells are stem cells or cancer cells. In an exemplary embodiment, the cells are connective tissue progeny. In an exemplary embodiment, the cells are mesenchymal cells. In an exemplary embodiment, the cells are induced pluripotent stem cells. In an exemplary embodiment, the cells are chondrocytes (e.g., derived from iPSCs/chondro-progenitor cells).

In certain embodiments, the cells are cultured in vivo or in vitro in the prepared three dimensional hydrogel.

The three dimensional hybrid hydrocolloid-hydrogel composition can be prepared by contacting (e.g., mixing) a fully dissolved, aqueous solution of a polycationic macromolecule with a fully dissolved, aqueous solution of a polyanionic macromolecule in a chamber whereby the three dimensional hybrid hydrocolloid-hydrogel is formed. In certain embodiments, the fully dissolved, aqueous solution of at least one polyanionic macromolecule comprises a suspension of cells. In certain embodiments, the fully dissolved, aqueous solution of the at least one polycationic macromolecule comprises a suspension of cells. In certain embodiments, the fully dissolved, aqueous solution of at least one polyanionic macromolecule comprises at least one therapeutic/biologically-active agent. In certain embodiments, the fully dissolved, aqueous solution of at least one polycationic macromolecule comprises at least one therapeutic/biologically-active agent.

In an exemplary embodiment, the three dimensional hybrid hydrocolloid-hydrogel composition has no detectable bubbles.

In an exemplary embodiment, the three dimensional hybrid hydrocolloid-hydrogel composition has PEC (polyelectrolyte complex) filaments originating at the interface of the polycationic and polyelectrolyte solutions and which extend into the suspension of cells.

In some embodiments, the stiffness of the hybrid gel composition may be controlled by the solvating fluid. In other embodiments, the low molecular weight dextran sulfate in the solvating fluid may be used to control the stiffness of the hybrid gel composition. In certain embodiments, the stiffness of the hydrogel may be modified by the Mw of the polyanionic macromolecule (e.g., hyaluronan). In certain embodiments, the stiffness of the hydrogel may be modified by the percentage of solids in the final construct. The stiffness may be measured by Young's modulus using any instrument known in the art. The stiffness of the hybrid gel composition influences the cellular response of the embedded cells. Different amounts of stiffness cause different cellular responses. The embedded cells retain mechanical information from the surrounding environment which can influence the embedded cells phenotype. A stiffness of about 0.25 kiloPascals (kPa) to about 1 kPa of the hybrid gel composition promotes neurogenesis. A stiffness of about 10 kPa of the surrounding environment from the hybrid gel composition promotes myogenesis. A stiffness of about 20 kPa of the hybrid gel composition promotes cartilage cell differentiation. A stiffness of about 30 kPa to about 50 kPa of the hybrid gel composition promotes osteogenesis. In some embodiments, the stiffness of the hybrid gel composition may be about 0.5 kPa to about 60 kPa. In some embodiments, the stiffness of the hybrid gel composition may be about 0.25 kPa, about 0.5 kPa, about 0.75 kPa, about 1 kPa, about 2 kPa, about 3 kPa, about 5 kPa, about 10 kPa, about 15 kPa, about 20 kPa, about 25 kPa, about 30 kPa, about 35 kPa, about 40 kPa, about 45 kPa, about 50 kPa, about 55 kPa, about 60 kPa, about 70 kPa, about 80 kPa, about 90 kPa, about 100 kPa, or a range between any of these values.

The hydrogels of the invention may be formulated for passage through narrow gauge needles, cannulas, and catheters of 18-27 gauge to expand therapeutic applications to transplantation of xenograft islets to surgically challenging locations such as subjacent to the hepatic capsule as well as to transplantation of stem cells, chondrocytes and therapeutic morphogens to the nucleus pulposus for intervertebral disc in treatment of degenerative disc disease (DDD).

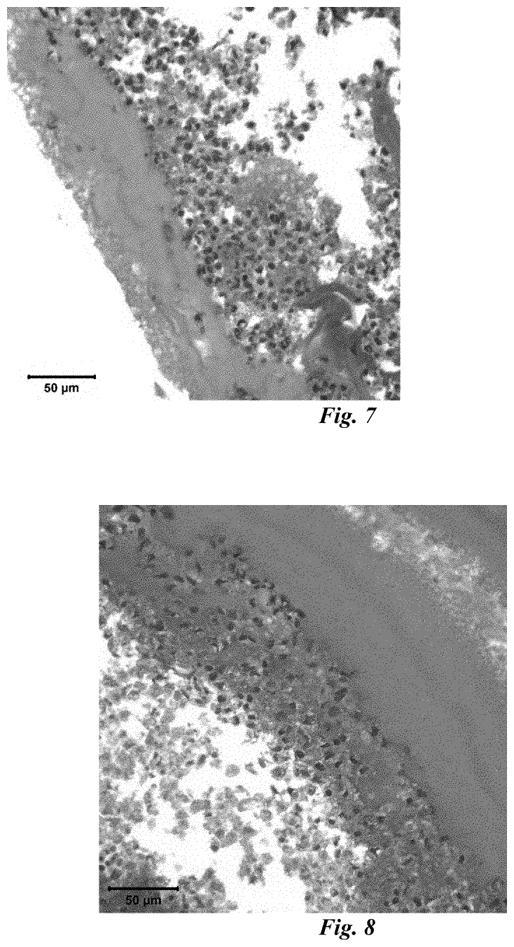

Hydrogels of the invention may be formulated to include non-structural compounds of the extracellular matrix (ECM) such as negatively charged fibronectin, vitronectin, laminin, collagen type II, collagen type IV, specific growth factors, morphogens and cytokines. These ECM components may be selected to work in concert with one another to direct pluripotent cells toward prescribed phenotypic destinations. An example of which is prescribed differentiation of iPSC (induced pluripotent stem cells) to the chondrocyte phenotype for use in treatment of articular cartilage defects and degenerative nucleus pulposus. Culture of iPSCs in a hydrogel of the invention is described in Examples 5-6 and shown in FIGS. 7 and 8.

Advantages of the hydrogel composition of the invention are afforded by contacting (e.g., mixing) the polycationic and polyanionic macromolecules after they have been fully dissolved in separate water based solutions. The advantages include: (1) improved control over cell distribution through the hydrogel three dimensional volume; (2) improved control over the hydrogel's mechanical properties, such as its stiffness over a wide range of Young's Modulus values; and (3) improved effectiveness of passive mass transfer. Mass transfer relates to the exchange of solutes in media surrounding the hydrogel such as nutrients and electrolytes with solutes of fluids inside the boundaries of the hydrogel. Passive mass transfer provides movement of nutrient solutes of surrounding media into the hydrogel while waste products generated by cells within the hydrogel boundaries are simultaneously removed where each class of solute moves in response to its respective concentration gradient.

Physical properties and performance functions of the hybrid hydrocolloid-hydrogel are controlled by manipulating certain constituent parameters, including:

(i) weight average molecular weight of the polycation and polyanion macromolecules;

(ii) charge density of the polycation and polyanion macromolecules;

(iii) charge ratio of the polycation and polyanion macromolecules;

(iv) concentration of the polycation and polyanion macromolecules in their respective (separate) solutions where concentration of solutes governs the solution viscosity;

(v) pH of polycation solution as it increases from room temperature to 37.5° C.;

(vi) order of addition for reactants;

(vii) methods for addition of reactions, including deposition of the second reactant on the surface of the first reactant, and injection of the second solution within the volume of the first solution; and

(viii) optional presence of a porous fabric such as Epi-Guide®, Curasan Inc. (de Santana, R B et al (2010) J Periodontol 81:926-933; U.S. Pat. Nos. 4,186,448; 5,133,755) to provide gross mechanical properties such as stiffness to optimize handling and shape of the hydrogel.

Further to these advantages, the hybrid hydrocolloid-hydrogel of the invention protects the DMEM (Dulbecco's Modified Eagle's Medium)/hyaluronic acid cell suspension from the adverse effects of acidity inherent in certain polycation macromolecules such as chitosan. By dissolving the polycationic and polyanionic macromolecules in separate solutions before mixing, entrapped air and resultant bubbles are avoided which interfere with and limit the utility of microscopy.

The hydrogel process of the invention allows for independent formulation of the cell suspension solution such that the extracellular matrix (ECM) may be customized and optimized for each cell line or cell type under study.

The hydrogel of the invention provides a method for monitoring cell behavior by observing by light microscopy a suspension of cells in a three dimensional hybrid hydrocolloid-hydrogel prepared by a process described herein. For example, by a process comprising contacting (e.g., mixing) a fully dissolved, aqueous solution of a polycationic macromolecule with a fully dissolved, aqueous solution of a polyanionic macromolecule comprising the suspension of cells in a chamber whereby the three dimensional hybrid hydrocolloid-hydrogel is formed.

In certain embodiments, the microscopy is confocal microscopy, epifluorescence microscopy, or inverted brightfield microscopy.

In an exemplary embodiment, the cells are stem cells or cancer cells. In an exemplary embodiment, the cells are connective tissue progeny. In an exemplary embodiment, the cells are mesenchymal cells. In an exemplary embodiment, the cells are induced pluripotent stem cells. In an exemplary embodiment, the cells are chondrocytes (e.g., derived from iPSCs/chondro-progenitor cells).

Cell migration, invasion, mitosis, cell shape changes, cell death, apoptosis, aggregation and cell clustering are cell behaviors which may be monitored by light microscopy of the hydrogels of the invention.

In certain embodiments, the hydrogel of the invention is free of large HA-CT-PEC fibers, which may cause optical interference.

FIG. 1 shows a scanning electron microscope (SEM) image of a cross-section of Epi-Guide® (Curasan Inc.) fabric showing graded pore volumes. The largest and least dense organization shown at top surface; smallest and most dense organization to bottom surface demonstrating a cell barrier function.

FIG. 2 shows a scanning electron microscope (SEM) image of Epi-Guide® (Curasan Inc.) fabric from the top surface with large pore configuration.

FIG. 3 shows a scanning electron microscope (SEM) image of Epi-Guide® (Curasan Inc.) fabric from the bottom surface with small pore configuration.

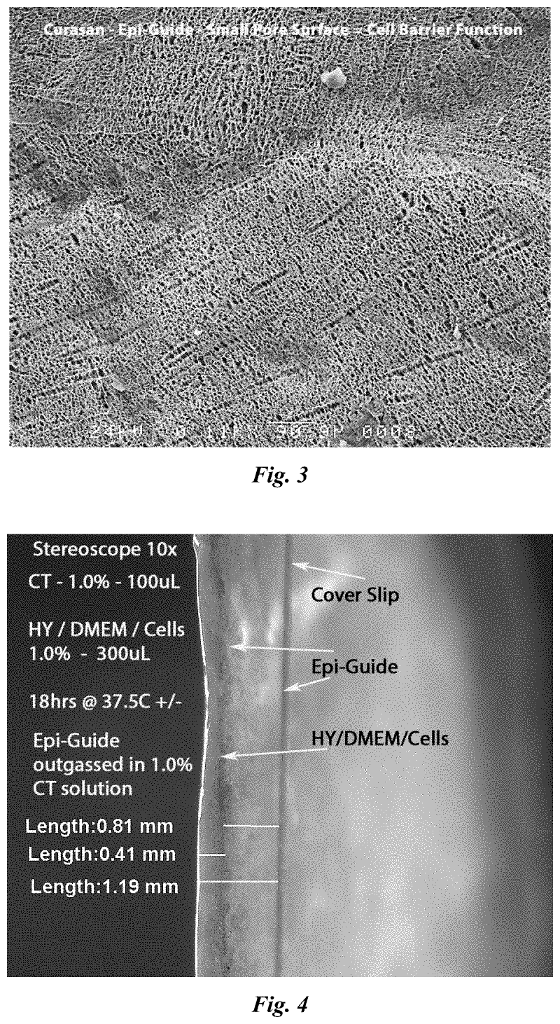

FIG. 4 shows a 10× microscope image of a hydrogel construct mounted vertically on a cover slip.

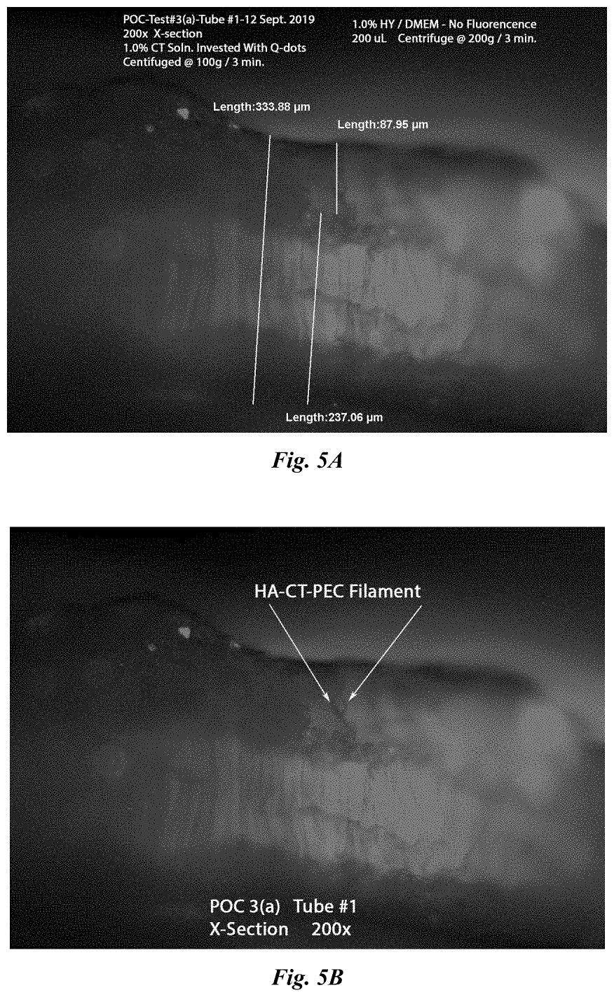

FIG. 5A shows a BX40-200x Epi-Fluorescence microscope image of at 610 nm. Chitosan treated with Q-dots fluorescing at 610 nm. DMEM-Hyaluronan does not fluoresce but is back-lit by fluorescence from adjacent Q-dots.

FIG. 5B shows a BX40-200x Epi-Fluorescence microscope image at 610 nm and the same image as shown in FIG. 5A, but annotated here to identify elements which may be HA-CT-PEC (hyaluronan-chitosan-polyelectrolyte complex) filaments originating at the interface of the chitosan solution. (within Epi-Guide® (Curasan Inc.) interstices) and DMEM/hyaluronic acid/cell suspension solution (laminated on surface of Epi-Guide®/chitosan complex).

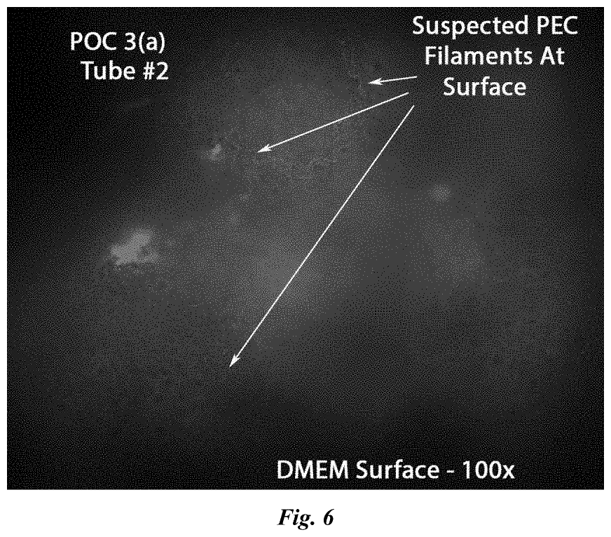

FIG. 6 shows a BX40-100x Epi-Fluorescence microscope image at 610 nm. The surface of the DMEM/hyaluronic acid/cell suspension hydrogel component shows a suspected projection of HA-CT-PEC filaments at the DMEM surface. FIG. 6 suggests that very thin, thread-like PEC filaments originate at the interface of the polycation and polyanion solutions and extend into the substance of the DMEM/hyaluronic acid cell suspension, hydrogel solution.

Cell behavior can be monitored by a method of observing by light microscopy a suspension of cells in a three dimensional hydrogel prepared by a process described herein. For example, by a process comprising contacting (e.g., mixing) a fully dissolved, aqueous solution of a polycationic macromolecule with a fully dissolved, aqueous solution of a polyanionic macromolecule comprising the suspension of cells in a chamber whereby the three dimensional hydrogel is formed.

In certain embodiments, the fully dissolved, aqueous solution of a polycationic macromolecule and the fully dissolved, aqueous solution of a polyanionic macromolecule are integrated by centrifugation after contact. In certain embodiments, centrifugation occurs shortly after these solutions are placed in contact with one another for example, within about 5, 10, 15, 20, 25, 30, 40, 50, or 60 mins after contact. In certain embodiments, the hydrogel construct is centrifuged at about 100 g, 150 g, 200 g, 250 g, 300 g, 350 g, or 400 g. In certain embodiments, the hydrogel construct is centrifuged for about 1, 2, 3, 4, 5, 6, 7, 8, 9, 10, 15, 20, 25, 30, 35, 40, 45, 50, 55 or 60 min. In certain embodiments, the hydrogel construct is centrifuged once. In certain embodiments, the hydrogel construct is centrifuged two or more times. In certain embodiments, the g-force and/or duration of the centrifugation is altered over several time intervals. Additionally, centrifugation may take place after the polyanion and polycation solutions are placed on top of a porous support/transwell to extract free water. As described herein, centrifugation may be used to increase the stiffness of the hydrogel.

In an exemplary embodiment, cells to be incorporated (within the forthcoming three-dimensional (3-D) microenvironment) are accumulated as a pellet and resuspended within a prescribed volume of the polyanion solution comprised of cell culture maintenance media (DMEM F12) containing 0.5-1.5% hyaluronan (wt/volume). This viscous solution is subsequently positioned on the membrane of a transwell insert and evenly distributed across its surface. The polycation (chitosan) solution (also of high viscosity) is placed (laminated) on the surface of the polyanion/cell suspension solution within the transwell insert. Integration of the two solutions begins by centrifugation immediately after they are placed in contact with one another in the transwell insert at a RCF (relative centrifugal force) of about 150 g for approximately 25 min.

Centrifugation is responsible for simultaneous introduction of 3 physical attributes important for formulation of the construct: A) reactants and attendant cells are mixed (blended) by agitation under “g” forces; B) polyanion and polycation macromolecules are concentrated together with attendant cells (by force of centrifuge as well as by simultaneous removal of dilute phase fluid) while they engage one another by (i) asymmetric coacervation (polyelectrolytic complexation—PEC), (ii) receptor/ligand binding and (iii) electrostatic attraction of cells to available chitosan molecules; C) water is separated from the colloidal construct into the adjacent dilute phase and removed from the system by centrifugal forces through the insert's bottom membrane.

In certain embodiments, the fully dissolved, aqueous solution of a polycationic macromolecule and the fully dissolved, aqueous solution of a polyanionic macromolecule are integrated by vacuum filtration after contact.

Concentrating reactant macromolecules improves mechanical properties of the coacervate by: (i) reordering polymer chain morphologies of the reactants, (ii) forming new (additional) electrostatic bonds as a consequence of this reorganization and (iii) initiating secondary and tertiary molecular bonds (e.g. hydrogen bonds and van der Waals interactions). Additionally, accomplishing these chemistries within a transwell insert allows for immediate evacuation of dilute phase water through the transwell membrane, thus preventing contamination of coacervate surfaces by adsorption of free (unreacted) polymers in the dilute phase onto their surfaces.

Applications of the hydrogels of the invention include a wide variety of biomimetic, three dimensional, in vitro, cell culture matrices, customizable to serve particular cell types. In particular, hydrogels of the invention may be useful for: (a) study and differentiation of stem cells; (b) in vitro modeling of malignant tumors and other disease processes; (c) treatment of intervertebral disc degeneration serving as a stand-alone repair modality for regeneration and/or replacement of the nucleus pulposus; (d) biomimetic carrier for delivery of reparative cells to regenerate the nucleus pulposus. Hydrogels of the invention may also be used for the delivery of therapeutic/biologically-active agents.

### Therapeutic Hydrogel Formulations

The three dimensional hydrogel compositions of the invention may be formulated with one or more therapeutic/biologically-active agents as therapeutic hydrogel formulations for the treatment of diseases and disorders. Beneficial or desired clinical results from treatment with the therapeutic hydrogel formulations include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. Treatment also includes prolonging survival as compared to expected survival if not receiving treatment of hyperproliferative diseases, such as cancer. Those in need of treatment include those already with the condition or disorder as well as those prone to have the condition or disorder or those in which the condition or disorder is to be prevented.

Therapeutic hydrogel formulations comprise a therapeutically effective amount of a therapeutic/biologically-active agent effective to treat a disease or disorder in a mammal. Therapeutic hydrogel formulations may be useful for treating a human or animal patient suffering from a disease or disorder arising from abnormal or hyperproliferative cell growth, an immune disorder, cardiovascular disease, viral infection, inflammation, a metabolism/endocrine disorder, a neurological disorder, a degenerative disorder including osteoarthritis, or traumatic injuries such as articular cartilage injuries. Therapeutic hydrogel formulations may be used to deliver therapeutic cells or other therapeutic/biologically-active agents to injury sites or in the case of cartilage injury may serve as a scaffold for endogenous cells to enter and repair the injured cartilage.

In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can, for example, be measured by assessing the time to disease progression and/or determining a response rate. Examples of cancer which may be treated with the therapeutic hydrogel formulations include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. In certain embodiments, the caner is ovarian cancer (e.g., metastatic ovarian cancer).

Therapeutic agents are active pharmaceutical ingredients with known drug-like properties, and include drugs approved by regulatory agencies such as the Food & Drug Administration or under clinical or pre-clinical study for specific effects in treatment of disease or disorders. Therapeutic agents may be biological “large molecules” such as antibodies, proteins, antibody-drug conjugates and the like, or chemical “small molecules” with a molecular weight of 1000 kDa or less. In certain embodiments, the therapeutic agent is a cytotoxic agent, such as cisplatin or paclitaxel. A biologically active agent includes but is not limited to a growth factor, cytokine, morphogen, extracellular vesicles/exosomes, mRNA, miRNA, lncRNA, and DNA.

In some embodiments, the therapeutic hydrogel formulation further comprises one or more pharmaceutically or pharmacologically-acceptable excipients, diluents and carriers as a pharmaceutical composition. The pharmaceutical compositions of the invention will be formulated, dosed and administered in a fashion, i.e., amounts, concentrations, schedules, course, vehicles and route of administration, consistent with good medical practice.

Therapeutic hydrogel formulation can be administered by any suitable means, including parenteral, intrapulmonary, and intranasal, and, if desired for local treatment, intralesional administration. Parenteral infusions include intramuscular, intravenous, intraarterial, intraperitoneal, intra-articular, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein. In some embodiments, the therapeutic hydrogel formulation can be delivered by parenteral administration, such as intravenous (IV) administration or administration into a body cavity or lumen of an organ. Alternatively, the therapeutic hydrogel formulation can be injected at a specific site, tissue, organ of a patient. Compositions for injection will commonly comprise the therapeutic hydrogel formulation dissolved in a pharmaceutically acceptable carrier.

The therapeutic hydrogel formulation may be delivered as a unit, solid oral dosage form in a pill, capsule, cachet or tablet for oral administration. Optimized solid oral dosage forms modulate release and pharmacokinetic profile, minimize the frequency of dosing, and minimize pill burden in patients with limited swallowing ability and other compliance factors.

### EXAMPLES

### Example 1. Injectable Formulation

The polycationic and polyanionic solutions are rendered sterile. Each is prepared separately under sterile conditions taking into consideration their respective molecular weights and ratios of their charge densities and mass. The two syringes are attached to opposite ends of a reciprocating adapter and the polycationic and polyanionic solutions are mixed via a reciprocating action between the two syringes. Following thorough mixing, the blended solution is injected into an in vitro cell culture container or into the target tissue/wound via a narrow gauge needle. (e.g. 23 ga needle). The polycationic and polyanionic solutions may be injected through a bioprinter following manufacturer protocols.

### Example 2. For In Vitro or In Vivo Applications Requiring Advanced Gross Mechanical Properties

1. All instruments, containers and solutions are rendered sterile.
2. Polycation compound(s) and polyanion compound(s) are selected for particular molecular weight and charge density values.
3. The polycationic solution is formulated by dissolving chitosan in sterile water (e.g. water for injection-WFI) at a concentration of about 1.0% (weight/volume) and passing it through a sterilization filter.
4. A biodegradable fabric made of D,D-L,L polylactic acid is commercially available from Curasan, AG, under the trade name of EpiGuide®. This fabric is immersed in the chitosan solution so that the polycationic solution is inculcated (invested/embedded/instilled, etc.) within its internal architecture of intercommunicating interstices. These void spaces are rank ordered by volume from largest at one surface to smallest at the opposite surface (see, FIGS. 1, 2, 3). The construct's entire volume of polycationic solution is contained within the void volume of the D,D-L,L polylactic acid (Epi-Guide®) material. The fabric thus prepared is relieved of excess solution and place on the bottom of a cell culture well or other cell culture apparatus.
5. In an exemplary embodiment, hyaluronan of molecular weight about 1.5×10⁶ (Lifecore Biomedical) is dissolved in cell culture base media at a concentration of about 1.0%. Hyaluronan may be used alone or in conjunction with a plurality of negatively charged, extra cellular matrix glycoproteins. This solution is used to resuspend cells of the cell pellet and deposited on the exposed surface of the polycation charged, polylactic acid (PLA) fabric.
6. The assembled construct is centrifuged for about 3 minutes at a force not exceeding 300 g and incubated for about 4 hours at 37.5° C. Fresh cell culture media is applied over the construct while incubation is continued as usual.

### Example 3. Differentiation of Human Mesenchymal Stem Cells (hMSCs) to Chondrocytes/Cartilage In Vitro in an Epi-Guide-Hydrogel Construct

Preparation of porous membrane invested with chitosan solution: see, Example 2.

Preparation of cells to be embedded in CT/HA hydrogel: Bone marrow-derived human mesenchymal stem cells (hMSCs, Sciencell, Carlsbad, Calif.) are expanded in plate culture according to the manufacturer's instructions, harvested with TrypLE® (Thermo Fisher Scientific), washed 2× in phosphate buffered saline, and counted. 28 million cells are then suspended in 0.7 ml of DMEM medium with 10% FBS and 1.0% sodium hyaluronan.

Generation of cell loaded Epi-Guide-Hydrogel construct: The 0.7 ml MSC suspension is carefully layered on top of the Epi-Guide invested with chitosan solution to allow contact between the chitosan and hyaluronan solutions to initiate formation of the cell-invested construct.

Culture of Epi-Guide-Hydrogel constructs: Constructs are placed in 6 well plates (1 per well) and cultured at 37° C. in 5% CO₂ in human MSC chondrocyte differentiation medium (Lonza) and medium changed as needed.

Analysis of Epi-Guide-Hydrogel constructs for differentiation of MSCs to cartilage: After 2 months of culture, samples are placed in 10% neutral buffered formalin solution and fixed at room temperature for 3.5 hours. Following fixation samples are transferred to 70% ethanol solution until processed for routine paraffin embedding. Samples are then sectioned 4 μm thick and stained with hematoxylin and eosin (H&E) and Alcian blue. For immunohistochemical staining, sections are cut at 4 μm, deparaffinized, and rehydrated, followed by incubation with 3% hydrogen peroxide to quench endogenous peroxidase activity and 15 min in serum free protein block (DAKO, Glostrup, Denmark). Sections are then subjected to appropriate antigen retrieval methods (if needed) and incubated with the primary antibody at room temperature for 60 min. Color development in the sections is done with EnVision® kits (Dako) per manufacturer's instructions. Stained sections are examined using a brightfield microscope.

Assessment: Sections of the Epi-Guide-Hydrogel constructs containing MSCs differentiated to cartilage are examined for evidence of differentiation by assessing 1) morphologic features on H&E stained sections (typical cartilage morphology includes rounded cells surrounded by abundant extracellular matrix with pale basophilic to pale eosinophilic coloration), 2) production of appropriate extracellular matrix with high levels of glycosaminoglycan content as demonstrated in Alcian blue stained sections, and expression of both cartilage protein markers type II collagen and aggrecan.

### Example 4. Fabrication of Three Dimensional Hydrogels

A 1.5% chitosan solution was made by dissolving chitosan (Primex) in water for injection (WFI) (Gibco). The solution was then passed through a sterilization filter (Pall, Life Sciences Acrodisc, PN4908).

Hyaluronan of molecular weight about 1.5×10⁶ (Lifecore Biomedical) was dissolved in DMEM at a concentration of 1.0%.

50 μL of the DMEM-Hy solution was deposited on the bottom of a first culture well and 75 μL of the chitosan solution was added on top. In a second well, 75 μL of the chitosan solution was deposited on the bottom and 50 μL of the DMEM-Hy solution was added on top.

The assembled constructs were centrifuged for 5 minutes at 100 g, 200 g or 300 g. 100 μL of fresh DMEM was added at various timepoints after centrifugation (e.g., 0-21 hr. post centrifugation). Addition of fresh media immediately after centrifugation was shown to dilute the hyaluronan and chitosan reactants, reducing the thickness and density of the final sheet construct.

At 36 hours, the added fresh DMEM was still pink (no evidence of excessive acidity).

### Example 5. Culture of Induced Pluripotent Stem Cells (iPSC) in Three Dimensional Hydrogel

A single pellet of 9.0×10⁶ iPSC, prepared from a 2-D population expansion culture, released by citrate buffer and centrifuged, was suspended in 50 μL (microliters) of 1% hyaluronan in DMEM (DMEM/1% Hy) and at 1% made up at least 7 days prior. The cell pellet volume was estimated at about 50 μL. The total volume of the cell suspension solution was about 100 μL. About 50 μL of the cell suspension solution was each used to set-up 2 wells in a standard, 8×12=96 well plate.

In Well #1, 75 μL of 1.5% chitosan solution was deposited on the well bottom. 100 μL of the cell suspension solution (50 μL of 4.5×106 iPSC cells+50 μL DMEM/1% Hy) was added on top for a total volume of 175 μL. A view under phase contrast microscope showed projection displaced up one side of culture well, presumably displaced cell suspension.

In Well #2, 100 μL cell suspension solution (50 μL of 4.5×106 iPSC cells+50 μL DMEM/1% Hy) was deposited on the well bottom. 75 μL of 1.5% chitosan solution was deposited on top for a total volume of 175 μL. A view under phase contrast microscope showed uniform distribution across the bottom of the well.

The constructs in Wells #1 and #2 were centrifuged at 100 g for about 5 minutes then placed in an incubator for 45 minutes for a total time of about 75 minutes from cell loading to microscopy.

200 μL of a standard solution of staining dyes calcein AM and ethidium bromide was applied to each well. The staining solution was allowed to penetrate the construct for about 5 minutes. The calcein AM/ethidium bromide staining solution was used to determine cell viability. Calcein AM is a cell-permeant dye that can be used to determine cell viability in most eukaryotic cells. In live cells the nonfluorescent calcein AM is converted to a green-fluorescent calcein after acetoxymethyl ester hydrolysis by intracellular esterases. Ethidium bromide is a nucleic acid intercalating agent which rapidly forms intense fluorescence upon binding to DNA.

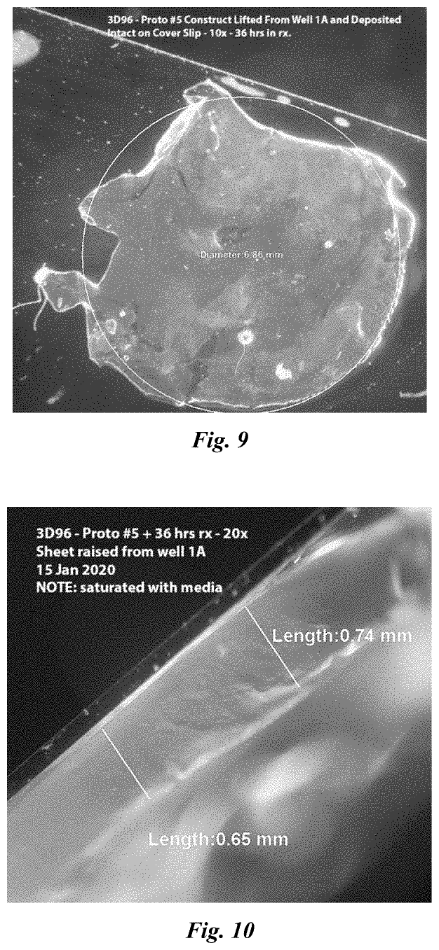

FIGS. 9 and 10 illustrate the materials fabrication principles in forming a three dimensional hydrogel. FIG. 9 shows a 10× microscopy image of iPSC cultured, lifted from Well #1A and deposited intact on a cover slip after 36 hours reaction. The construct is translucent after saturation with DMEM. FIG. 10 shows a 20× microscopy image of iPSC cultured, of a sheet raised from Well #1A, saturated with media after 36 hours reaction.

### Example 6. Culture of iPSC in Three Dimensional Hydrogel with Addition of Fresh Media

Hydrogels were prepared in a manner similar to that described in Example 5. Briefly, citrate buffer was used to release ˜36×10⁶ iPSC from a 2-D population expansion culture. These cells were pelleted by centrifugation and resuspended in a volume of DMEM/1.0% hyaluronan, sufficient to deliver 50 μL containing 4.5×10⁶ cells to each of 8 samples being established in a 96 well plate.

In all samples, the DMEM/hyaluronic acid/cell suspension solution was deposited on the bottom of the culture well as a 1^{st} step. 75 μL of the 1.5% sterile chitosan solution was subsequently deposited over (on top of) the 50 μL DMEM/hyaluronic acid/cell suspension solution. The plate was centrifuged at 100 g for 5 min. and incubated for ˜40 min. 100 μL of fresh DMEM was added to four of the samples.

Samples were evaluated for viability after 19 and 72 hours of incubation using a standard calcein-AM/ethidium bromide stain solution (see, Example 5). The samples having received supplemental media showed excellent viability and had more viable cells than the samples that did not receive supplemental media.

Microscopy of samples showed that cells successfully retained boundaries of the construct, which had varying degrees of retention of their original geometries. Construct stiffness may be increased as needed by, e.g., increasing the Mw of hyaluronan; increasing the percentage of solids in the final construct; and/or increasing the “g” forces and/or duration of the centrifugation (e.g., ramping or “g-forces” over several time intervals).

H&E staining (hematoxylin and eosin) was performed at day 7. FIGS. 7 and 8 show images after H&E staining at short term duration (FIG. 7) of less than 60 minutes out of fresh media and long term duration of greater than 60 minutes out of fresh media. FIG. 7 shows a microscopy image of iPSC cultured in a DMEM/hyaluronic acid/cell suspension hydrogel and H&E stained (hematoxylin and eosin) less than 60 minutes out of fresh media. FIG. 8 shows a microscopy image of iPSC cells cultured in a DMEM/hyaluronic acid/cell suspension hydrogel and H&E stained (hematoxylin and eosin) greater than 60 minutes out of fresh media according to Example 6.

Although the foregoing has been described in some detail by way of illustration and example for purposes of clarity of understanding, the descriptions and examples should not be construed as limiting the scope of the present disclosure. Accordingly, all suitable modifications and equivalents may be considered to fall within the scope of the present disclosure as defined by the claims that follow. The disclosures of all patent and scientific literature cited herein are expressly incorporated in their entirety by reference.

What is claimed is:

1. A process for preparing a three dimensional hydrogel embedded with cells comprising contacting a fully dissolved, aqueous solution of at least one polycationic macromolecule with a fully dissolved, aqueous solution of at least one polyanionic macromolecule comprising a suspension of cells in a chamber, whereby the three dimensional hydrogel is formed.

2. The process of claim 1 wherein the suspension of cells is a cell culture comprising one or more extracellular matrix compounds selected from the group consisting of fibronectin, vitronectin, laminin, collagen type I, chondoitin sulfate, collagen type II, hyaluronan, dextran sulfate and a soluble signaling factor selected from growth factors, morphogens and cytokines.

3. The process of claim 1 wherein the chamber contains a porous fabric.

4. The process of claim 3 wherein the at least one polycationic macromolecule fills interstices of the porous fabric.

5. The process of claim 4 wherein the porous fabric is selected from the group consisting of D,D, L,L polylactic acid, Epi-Guide, gelatin, methacrylate polymer, type I or type II collagen, hyaluronan, polyester, polycarbonate, poly(α-hydroxy) acid and polyethylene glycol.

6. The process of claim 1 wherein the polycationic macromolecule is chitosan.

7. The process of claim 1 wherein the polyanionic macromolecule is selected from the group consisting of hyaluronic acid, type I collagen, type II collagen, and dextran sulfate.

8. The process of claim 1 wherein the fully dissolved, aqueous solution of at least one polyanionic macromolecule comprising a suspension of cells further comprises a cell culture media selected from DMEM or other suitable base media.

9. The process of claim 1 further comprising centrifuging or vacuum filtering the three dimensional hydrogel positioned above a porous support to remove free water.

10. The process of claim 1 wherein the fully dissolved, aqueous solution of at least one polyanionic macromolecule and/or the fully dissolved, aqueous solution of at least one polycationic macromolecule further comprises a therapeutic/biologically-active agent.

11. The process of claim 1 wherein the fully dissolved, aqueous solution of the at least one polycationic macromolecule is deposited on the bottom surface of the chamber followed by adding the fully dissolved, aqueous solution of the at least one polyanionic macromolecule comprising a suspension of cells.

12. The process of claim 11 further comprising depositing a polycationic macromolecule to form a thin, dry film on the bottom surface of the chamber before contacting a fully dissolved, aqueous solution of at least one polycationic macromolecule with a fully dissolved, aqueous solution of at least one polyanionic macromolecule comprising a suspension of cells in the chamber, wherein the thin, dry film is optionally treated with fluorescent Q-dots.

13. The process of claim 11 wherein the aqueous solution of the at least one polycationic macromolecule is contacted with a thin, reinforcing matrix selected from D,D-L,L polylactic acid, collagen, and hyaluronic acid before adding the fully dissolved, aqueous solution of the at least one polyanionic macromolecule comprising a suspension of cells.

14. The process of claim 1 wherein the fully dissolved, aqueous solution of the at least one polyanionic macromolecule comprising a suspension of cells is deposited on the bottom surface of a chamber followed by adding the fully dissolved, aqueous solution of the at least one polycationic macromolecule.

15. The process of claim 14 wherein further comprising depositing a polyanionic macromolecule to form a thin, dry film on the bottom surface of the chamber, before contacting the fully dissolved, aqueous solution of at least one polycationic macromolecule with the fully dissolved, aqueous solution of at least one polyanionic macromolecule comprising a suspension of cells in the chamber, wherein the thin, dry film is optionally treated with fluorescent Q-dots.

16. The process of claim 1 wherein the cells are selected from the group consisting of stem cells, cancer cells, mesenchymal cells, and progeny thereof.

17. The process of claim 1 wherein the cells are induced pluripotent stem cells or chondrocytes.

18. A three dimensional hydrogel prepared the process of claim 1.

19. The three dimensional hydrogel of claim 18 having no detectable bubbles.

20. The three dimensional hydrogel of claim 18 wherein PEC filaments originate at the interface of the polycationic and polyanionic solutions and extend into the suspension of cells.

21. The three dimensional hydrogel of claim 18 having a Young's Modulus value of about 0.5 to about 60 kiloPascals.

22. A method of three dimensional cell culture comprising growing the cells in the three dimensional hydrogel of claim 18.

23. A method for monitoring cell behavior comprising observing by light microscopy the suspension of cells in the three dimensional hydrogel of claim 18.

24. A method of treating a disease, disorder or condition in a mammal in need thereof, comprising administering to the mammal the three dimensional hydrogel of claim 18.

25. The method of claim 24, wherein the disease, disorder or condition is selected from the group consisting of cancer, degenerative disc disease (DDD), traumatic articular cartilage injuries, osteoarthritis, periodontal defects, bone defects, and diabetes mellitus.

26. A process for preparing a three dimensional hydrogel comprising at least one therapeutic/biologically-active agent, the process comprising contacting a fully dissolved, aqueous solution of at least one polycationic macromolecule; a fully dissolved, aqueous solution of at least one polyanionic macromolecule; and optionally, a porous fabric; in a chamber, whereby the three dimensional hydrogel is formed, wherein the porous fabric, the fully dissolved, aqueous solution of at least one polycationic macromolecule and/or the fully dissolved, aqueous solution of at least one polyanionic macromolecule comprises at least one therapeutic/biologically-active agent.

27. A three dimensional hydrogel prepared by the process of claim 26.

28. A method of treating a disease, disorder or condition in a mammal in need thereof, comprising administering to the mammal the three dimensional hydrogel of claim 27.
